(19) United States
(12) Patent Application Publication
(10) Pub. No.: US 2023/0137547 A1
(43) Pub. Date: May 4, 2023

(54) Solid Deep Eutectic Solvent Formulation Platform

(71) Applicant: SeraNovo Holding B.V., Leiden (NL)

(72) Inventors: Christian Degeling, The Hague (NL); Niall David Hodgins, Leiden (NL); Bastiaan Kluft, Leiden (NL)

(21) Appl. No.: 17/905,949

(22) PCT Filed: Mar. 9, 2021
(86) PCT No.: PCT/NL2021/050158

§ 371 Date: Sep. 9, 2022

(30) Foreign Application Priority Data
Mar. 10, 2020 (NL) 2025092
Feb. 19, 2021 (NL) 2027606

(51) Int. Cl. A61K 31/185; A61K 47/10; A61K 47/69; A61K 47/18; A61K 9/00; A61K 47/58

(52) U.S. Cl. CPC A61K 31/185; A61K 9/0019; A61K 47/10; A61K 47/58; A61K 47/69; A61K 47/183; A61K 47/186

(57) ABSTRACT

The invention relates to a solid deep eutectic solvent (solid DES) that is solid at 20° C., comprising a salt of an active pharmaceutical ingredient, the method to obtain the solid deep eutectic solvent and the use of the deep eutectic solvent for medical treatment. The solid deep eutectic solvent is obtained by at least partially removing a plasticizer from a first DES.

The invention is in the field of deep eutectic solvents. In particular the invention relates to a method to prepare a solid deep eutectic solvent (DES) that is solid at 20° C. (herein also referred to as solid DES), from a deep eutectic solvent comprising an active pharmaceutical ingredient (API). The invention further relates to solid deep eutectic solvent obtainable by this method.

A deep eutectic solvent (DES) is formed when one or more constituents are mixed together at a specific ratio resulting in the depression of the melting point of one or more of the constituents. The deep eutectic phenomenon was first described for a mixture of choline chloride and urea in a 1:2 molar ratio, respectively.

A DES shares a lot of characteristics with an ionic liquid, however, a DES is typically an ionic mixture and not a single, individual ionic compound. Where ionic liquids require a chemical reaction, a DES is typically merely a physical mixture. A DES may be non-flammable, biodegradable and it may have a higher than water density. Moreover, a DES generally has the ability to dissolve many metal salts and organic compounds. Furthermore, a DES is typically relatively cheap to make in contrast to ionic compounds. It may be used as a safe, efficient and low-cost solvent.

In the pharmaceutical industry, a challenge remains on the solvation of poorly water-soluble APIs and their bioavailability. Advanced formulations are often necessary in order for the API to be effectively absorbed in the gastrointestinal (GI) tract. The terms “API”, “drug”, “active ingredient” may be used interchangeably herein.

The bioavailability of the API typically i.a. relates to the solvability, membrane permeability and enzymatic degradation of the active ingredient in the patient. Bioavailability is typically denoted as a percentage and measured as the area under curve of the plasma concentration of a drug collected over a certain period of time, which depends i.a. on the half-life of the drug. As most of the APIs are poorly soluble in water, handling of these ingredients often require the use of less polar solvents such as dimethylsulfoxide, alcohols, acetone, ethyl acetate, chloroform and the like. These solvents present problems including toxicity and danger of explosion.

Several alternatives for enhancing the solubility of APIs have been proposed in the form of physical modifications, such as solid dispersions or chemical modifications, such as salt formation.

Salt formation is a tool to obtain solid-state forms as the ionic interactions in a crystal lattice may be beneficial to crystal lattice stabilization. The ion-ion interactions, moreover, typically contribute strongly to the lattice enthalpy. However, a drawback of salt formations is the tendency to form hydrates as the ions within the salt interact strongly with water. Thereby decreasing stability and limiting shelf life. In addition, not all APIs can be formulated in salts.

Solid dispersions are dispersions of a drug in a typically amorphous polymer matrix. The drug is preferably molecularly dispersed in the matrix. The matrix may further comprise additional additives such as surfactants. Solid dispersions can be manufactured via a plurality of processes including melt extrusion, spray drying and co-precipitation. Several reviews on solid dispersions are Huang and Dai (Acta Pharmaceutica Sinica B, 4, 2014, 18-25), Chivate et al. (Current Pharma Research, 2, 2012, 659-667) and Zhang et al. (Pharmaceutics, 3, 2018, 142). Disadvantages of solid dispersions include the lack of a uniform manufacturing process and the instability of the product. Moreover, a decrease in dissolution rate is observed over time.

Another example of a method to attempt to improve the solubility of poorly soluble APIs is disclosed in WO98/55148. Herein a composition comprising a no more than sparingly water-soluble drug compound, a cyclodextrin, a physiologically tolerable water-soluble acid and a physiologically tolerable water-soluble organic polymer is described. The use of similar cyclodextrin-water soluble polymer ternary complexes for the API itraconazole is described in Taupitz et al. (European Journal of Pharmaceutics and Biopharmaceutics, 83, 2013,378-387).

The solvation and bioavailability of APIs may be improved by using a DES. DESs have demonstrated to substantially improve the bioavailability of poorly soluble active ingredients. Moreover, DESs may be biodegradable, safe and low-cost.

Aroso et al. (European Journal of Pharmaceutics and Biopharmaceutics, 98, 2016, 57-66) present a study of several DES formulations. The API in the DES showed a higher dissolution rate in comparison with the API alone. Furthermore, it was shown that the dissolution rate can be modified by selecting the hydrogen bond acceptor.

The application PCT/NL2020/050515discloses a DES using an API salt as one of its constituents. As the API salt is one of the constituents, the solubility and bioavailability of the API are further enhanced.

WO 2020/085904 discloses a DES platform for oral pharmaceutical formulations that also enhances the solubility and bioavailability of the API by designing a liquid in which a full dose of API can be dissolved. A common drawback is found in the limited stability and shelf life of a liquid DES with an API.

It is an object of the present invention to provide a DES that at least in part overcomes the above-mentioned drawbacks. In particular, it is an object of the present invention to provide a drug formulation with improved stability, an improved dissolution rate, and allowing a high amount of the API in the drug formulation. The present inventors have surprisingly found that using a salt of an API (herein also referred to as API salt) as a eutectic constituent in a DES allows a particular high API loading in the DES without compromising the stability of the DES. Further, the present inventors have surprisingly found that the stability of a DES comprising a API salt can be enhanced by at least partially removing a first plasticizer from a first DES to provide a solid DES that is solid at 20° C. Without wishing to be bound by theory it is believed that addition of the first or a second plasticizer to the solid DES forms the first or a second DES, respectively. It is further believed that this can occur in situ, after the administration of the solid DES to the body as bodily liquids including water may serve as the first and/or second plasticizer. Thus the excellent solubility and bioavailability of the API salt as demonstrated for DESs is retained in the present invention.

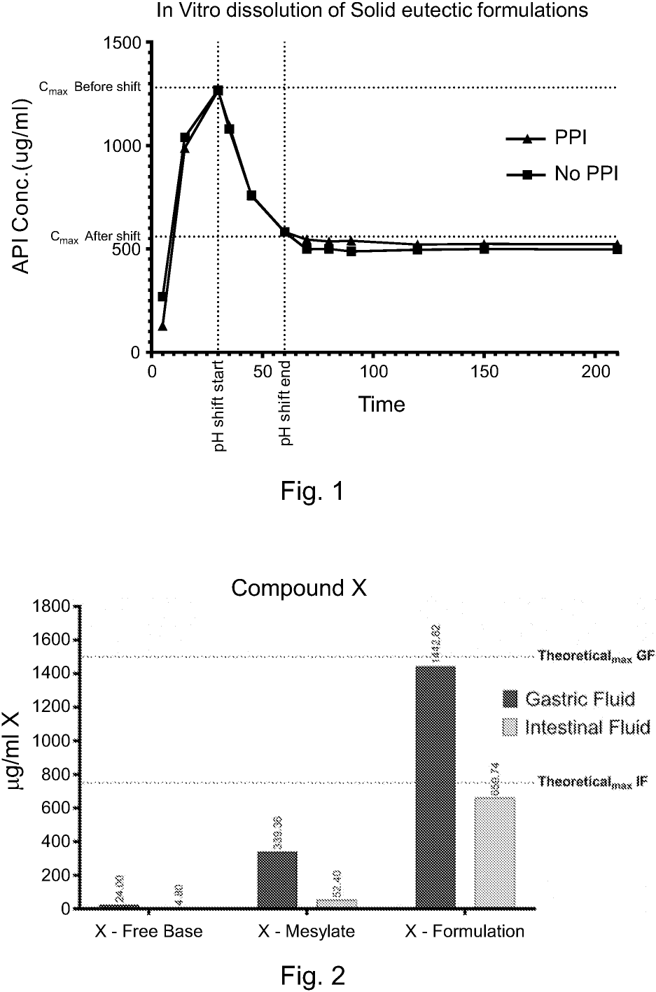

FIG. 1 illustrates an in vitro dissolution of two solid DESs, one comprising a PPI and one without a PPI.

FIG. 2 illustrates a comparative in vitro dissolution of a solid DES formulation comprising API X compared to the crystalline free base form of the API and the crystalline mesylate salt form of the API.

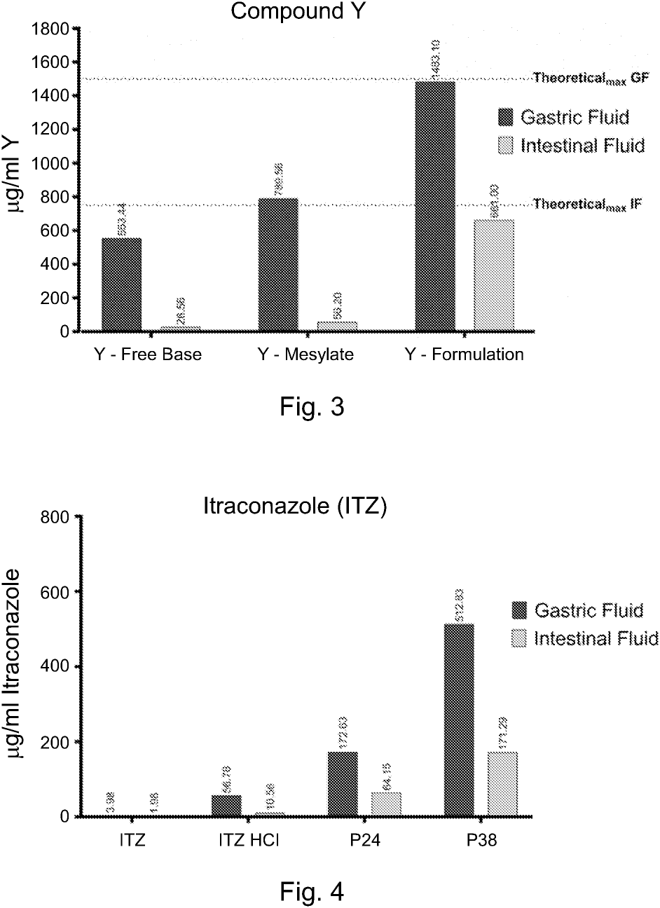

FIG. 3 illustrates a comparative in vitro dissolution of a solid DES formulation comprising API Y compared to the crystalline free base form of the API and the crystalline mesylate salt form of the API.

FIG. 4, a comparative in vitro dissolution of itraconazole HCl salt compared to its free base form and two solid DES formulations are illustrated.

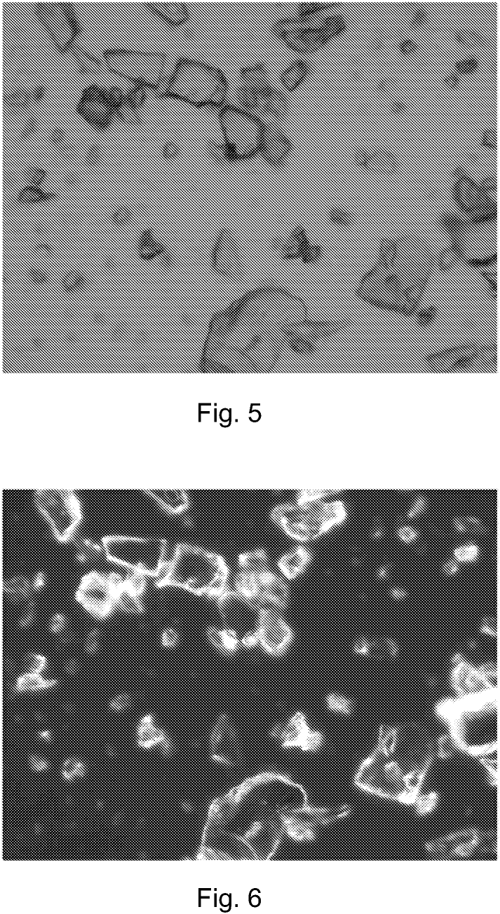

FIG. 5 is a polarized light microscopy image that illustrates that the solid DES is a glassy solid.

FIG. 6 is a polarized light microscopy image that illustrates that the solid DES is a glassy solid.

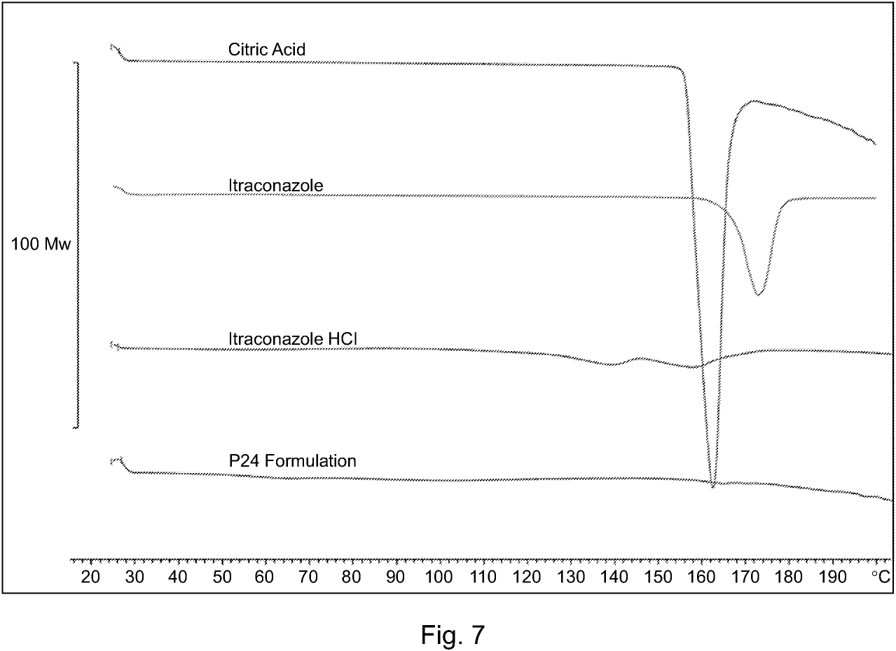

FIG. 7 shows four differential scanning calorimetery thermograms that indicate that the solid DES has no melting peak and thus lacks crystalline material and is therefore likely to be an glass.

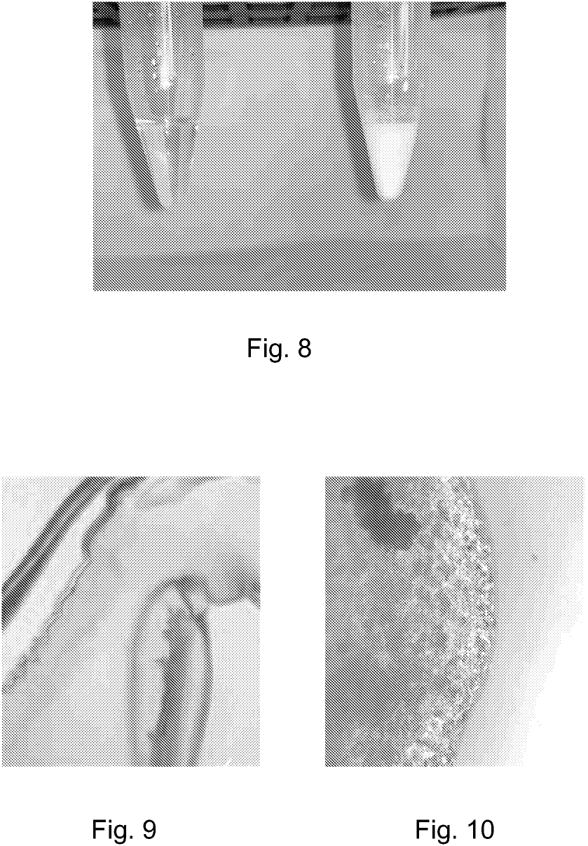

FIG. 8 illustrates a first DES comprising sildenafil citrate and urea compared to a control sample not comprising urea.

FIG. 9 is a polarized light microscopy image illustrating a solid DES based on a first DES comprising sildenafil citrate and urea.

FIG. 10 is a polarized light microscopy image illustrating a solid control sample based on a liquid control sample not comprising urea.

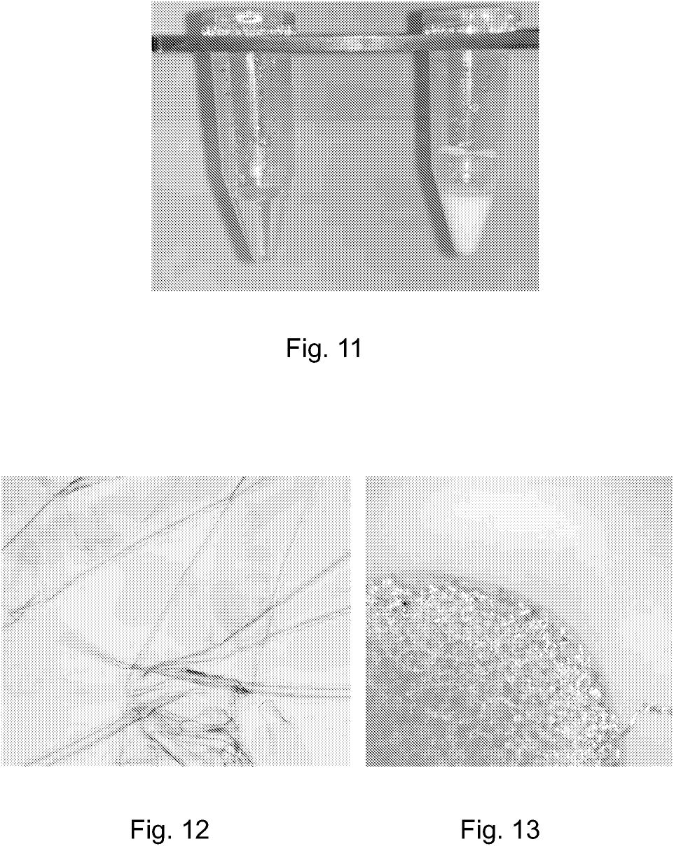

FIG. 11 illustrates a first DES comprising sildenafil citrate and ascorbic acid compared to a control sample not comprising ascorbic acid.

FIG. 12 is a polarized light microscopy image illustrating a solid DES based on a first DES comprising sildenafil citrate and ascorbic acid.

FIG. 13 is a polarized light microscopy image illustrating a solid control sample based on a liquid control sample not comprising ascorbic acid.

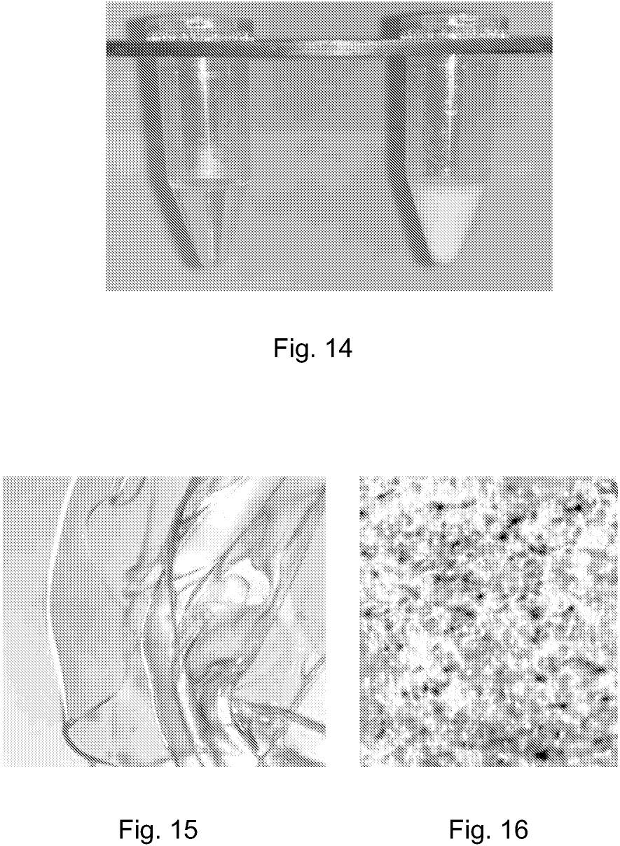

FIG. 14 illustrates a first DES comprising fexofenadine.HCL and meglumine compared to a control sample not comprising meglumine.

FIG. 15 is a polarized light microscopy image illustrating a solid DES based on a first DES comprising fexofenadine.HCl and meglumine.

FIG. 16 is a polarized light microscopy image illustrating a solid control sample based on a liquid control sample not comprising meglumine. Thus in a first aspect, the present invention is directed to a method to prepare a solid deep eutectic solvent (DES) that is solid at 20° C., said method comprising providing a first DES and partially removing a first plasticizer from said first DES, wherein said first DES comprises said first plasticizer, an active pharmaceutical ingredient (API) salt, a pharmaceutically acceptable eutectic constituent and optionally a polymeric precipitation inhibitor (PPI). It combines the advantages of the solubility and bioavailability enhancement of the DES with the higher stability benefits of solid formulations.

The solid DES is solid at 20° C., which inherently means that it is also solid at all temperatures below 20° C. The solid DES is typically stored at ambient temperatures such as 20° C., at which it is solid. Preferably the solid DES is solid up to 30° C., even more preferably up to 37° C. It is preferred to remain solid at slightly elevated temperatures to ensure sufficient stability during storage in slightly deviating environments. Furthermore, it is preferred that the solid DES remains solid up to body temperature as it may increase the effectiveness after administration and thereby improve the bioavailability.

As used herein, all temperatures are at 1 atm, unless specified otherwise.

The solid DES in accordance with the present invention is typically a eutectic mixture based on at least the API salt and the pharmaceutically acceptable eutectic constituent as the eutectic constituents. Accordingly, the melting point of the solid DES may be lower than at least that of the API salt and the pharmaceutically acceptable eutectic constituent. In case that the API salt itself is a constituent of the eutectic mixture, it is not required that the solid DES according to the invention is capable of being or used as a solvent.

The first DES may be obtained via conventional methods, wherein all constituents (i.e. the API salt and the pharmaceutically acceptable eutectic constituent) are mixed in their specific ratios (e.g. including heating until a liquid is formed). The obtained liquid is generally a stable liquid at 20° C., meaning that is it transparent for at least 24 h at 20° C. Suitable constituents for the DES can be selected, for example, for their hydrophilicity, melting point and viscosity. In addition, they may typically be selected for being safe for oral administration, for example they may be on the generally recognized as safe (GRAS) list for oral administration. The GRAS list is established by the Food and Drug Administration (FDA) of the United States. Furthermore, they may be selected for their ability to hydrogen bond with either the plasticizer, other DES constituents and/or the active ingredient, wherein the active ingredient may be ionized or a salt. The specific ratio between the constituents can be tuned in order create a stable DES.

The term “partially removing” herein means removing the first plasticizer from the first DES to the extent to at least yield the solid DES. The removal of the first plasticizer may thus be extended further to remove more plasticizer than necessary to yield the first solid DES.

One of the constituents on which the solid DES according to the present invention is based is the API salt. APIs are typically poorly water-soluble compounds and administration to a patient can be a challenge. The API may for example have an aqueous solubility of not more than 1 mg/mL at pH 6.8 when it is a weakly basic compound, of no more than 1 mg/mL at pH 1.2 when it is a weakly acidic compound. In the case of physiological pH of 1.0 to 8.0 for neutral or non-ionizable compounds the aqueous solubility may be no more than 1 mg/mL. A drug is considered highly soluble when the highest dose strength is soluble in 250 ml or less of aqueous media over the pH range of 1 to 7.5. If it does not dissolve in 250 ml at each of the aforementioned conditions, it is generally considered poorly water-soluble. An extensive list of possible APIs is mentioned in Pharmacopeia. The dosage for therapeutically effective amounts for a given API is typically known to a person skilled in the art. The term “aqueous environment” used herein generally relates to gastrointestinal fluid at pH 5.0 to 8.0 or to the stomach at pH 1.0 to 2.0 in vivo or an aqueous test medium in vitro.

API salt herein means that the API is ionized and combined with a counter ion, e.g. as a HCl salt of a basic active ingredient or as a Na salt of an acidic active ingredient. The fact that the API salt is ionized and combined with a counter ion means that the ionized API is not dissociated from the counter ion such as dissolved in a solution. The salt form of the API in accordance with the present invention is preferred over a dissociated ionized form of the API because it gives a more stable first DES and a more stable solid DES. When an ionized API that is dissociated from its counter ion is used, for example as described in Taupitz et al. (European Journal of Pharmaceutics and Biopharmaceutics, 83, 2013,378-387), this does not form a stable DES as the API will precipitate out of the composition over time. Without wishing to be bound by theory this precipitation is believed to be due to the common ion effect and/or due to degradation of the API.

Basing the first DES on the API salt can be achieved by providing the API salt as such, i.e. ex situ prepared, and mixing said API salt with the pharmaceutically acceptable eutectic constituent and the plasticizer and optionally the PPI. This method differs from in situ forming the API salt (e.g. by mixing a basic API with an acid). It was found that by ex situ preparing the API salt and basing the first DES on the API salt results in a more stable solid DES.

Advantageously, the API salt has an increased solubility and dissolution rate with respect to the non-ionized or free-base form of the API. The dissolution is generally enhanced by the counter ion of the salt as it changes the pH at the dissolving surface of a salt particle in the diffusion layer, thereby creating a higher dissolution rate compared to the corresponding free form of the API. The solubility is typically increased as is demonstrated by the Henderson-Hasselbalch equations. These indicate that the change of pH influences the aqueous solubility of an ionizable drug.

Moreover, the counter ion may interact strongly with an electron donor or acceptor group in the other constituents making up the first DES. Without wishing to be bound by theory it is believed that a small (e.g. atomic) counter ion such as Cl in HCl can facilitate strong intermolecular forces required to depress the melting point and create a DES. Therefore, preferably the API salt has a small counter ion capable of forming such strong interactions. Accordingly, in embodiments wherein the API salt is based on a basic API, the acid which with the API salt is formed preferably comprises an atomic anion (e.g. a halide anion), while said pharmaceutically acceptable eutectic constituent comprises one or more functional groups capable of hydrogen bonding. Alternatively, in the embodiment wherein the API salt is based on acidic API and a base that comprises a molecular anion, and said pharmaceutically acceptable eutectic constituent comprises an atomic anion or a small (e.g. atomic) moiety capable of forming strong intermolecular bonds. Formation of a eutectic mixture of an API salt and principles behind this concept is also described in PCT/NL2020/050515, which is incorporated herein in its entirety.

Further benefits of using an API salt include the self buffering capacity of the salt. This for instance allows for the salt to show resistance to precipitate out of the first DES. The buffering capacity may further enhance the dissolution rate of the API. Additionally, the API salt may allow for an overall more hydrophilic solid DES, this is for instance due to the coulombic interactions that may promote water uptake. A more hydrophilic solid DES may also contribute to a faster dissolution rate.

Further, a higher drug loading can be achieved when using an API salt. It is preferred that the change from the solid DES to a first or second DES is considered congruent with respect to the API salt and the pharmaceutically acceptable eutectic constituent. With this it is meant that during the change, the components (i.e. the API salt and the eutectic constituent) in the solid from are the same as in the liquid form. The API salt may allow for higher drug loading while the phase change is still considered congruent with respect to the API salt and the pharmaceutically acceptable eutectic constituent. This is as the API salt typicaly allows for optimal mixing at a molecular level, which allows for a smoother dissolution.

Accordingly, advantageously, the amount of the API salt in the solid DES is preferably more than 5 wt%, more preferably more than 10 wt%, most preferably 20 wt% or higher. The amount of API salt in the solid DES is typically limited by the amount of API salt that is required for it to form a eutectic mixture with the eutectic constituent. Typically, the weight ratio of the API salt and the eutectic constituent is in the range of 5:1 to 1:5, preferably in the range of 3:1 to 1:3, more preferably in the range of 2:1 to 1:2.

Another constituent on which the first DES according to the present invention is based is the plasticizer. The plasticizer is typically responsible for decreasing the plasticity and/or viscosity of a material and to enhance the thermal stability. The plasticizer is generally liquid at ambient temperature. Plasticizers usually do not form chemical bonds and fulfill their function mostly through intermolecular forces such as hydrogen bonds. The plasticizer differs in its role from for example a solvent in that a solvent is capable of solubilizing one or more solid components (i.e. solutes) only when the solvent is used in such an amount to provide an undersaturated solution of these components or to provide a solution at the solubility of these components.

In the present invention, the concentration of the API contained in the first DES can be greater than the maximum solubility of the API in the plasticizer istelf (i.e. in its isolated form and not combined with the eutectic constituent). In other words, the plasticizer may be used in an amount that it would be insufficient to dissolve the amount of API salt contained in the DES in an isolated form. In yet other words, the amount of the API salt in the first DES can be more than would be soluble in its isolated form in the amount of the first plasticizer that is used in the first DES. I.a. as such, the plasticizer differs from a conventional solvent. This is advantageous because it reduces the volume of plasticizer needed and the interactions between the API salt and the eutectic constituent prevents the API salt from precipitating before or during drying. For instance, the amount of the API salt in the first DES may be more than 2.5%, such as more than 5% based on the total weight of the first DES.

The plasticizer may be added from 10% (m/m) up to 60% (m/m), preferably from 15% (m/m) up to 40% (m/m), where % (m/m) relates to the mass of the plasticizer compared to the total mass of the first DES.

DESs work through the formation of a hydrogen bonding network and thus preferably, the plasticizer functions as a hydrogen bond donor and/or acceptor, more preferably the plasticizer functions as hydrogen bond donor. The plasticizer may have a low melting point and may assist the formation of the DES at lower temperatures than when no plasticizer is present. Furthermore, the plasticizer is typically miscible with water. The plasticizer typically fulfills its function by creating hydrogen bonds with the other DES constituents and lowering the melting point of the mixture. Groups that are typically good at forming hydrogen bonds are for example =O, OH, NHx, N=R, conjugated systems, acidic/basic groups and in general electronegative groups (F, O, Cl, N, Br, I, S, C, H) with a dipole moment.

Moreover, as the plasticizers are partially removed from the first DES to form the solid DES, the plasticizers are ideally easily removable by conventional methods such as evaporation. Other plasticizer removal techniques may include lyophilization, centrifugal evaporators, vacuum ovens, slow evaporation without applied vacuum, convective drying, drum drying, supercritical drying, dielectric drying and the like. Intrinsic properties such as volatility, average molecular weight and boiling points typically determine the ease of removal.

For an easy removal of the plasticizer by evaporation or lyophilation, the boiling point of the plasticizer is preferably below 250° C., more preferably below 150° C. at atmospheric pressure. Accordingly, the plasticizer is preferably a volatile plasticizer. Suitable plasticizers may include, but are not limited to, water, esters and lactones of organic acids, dicarboxylic acids and esters of dicarboxylic acids, desters, ethers and carbonates of diols and triols and mixtures thereof. These classes may include, but are not limited to, ethanol, glycerol carbonate, propylene carbonate, ethyl lactate, diethyl succinate, 1,2-hexanediol, 1,2-butylene carbonate, glycerol formal, 1-butoxypropan-2-ol, tri(propylene glycol)methyl ether, dipropylene glycol, triethyl citrate, methyl ether acetate, dimethyl acetamide, propylene glycol methyl ether acetate, dipropylene glycol methyl ether, 1-methoxy-2-propanol, diethylene glycol monoethyl ether, 3-methoxy-3-methyl-1-butanol, isosorbide dimethyl ether.

Preferably the plasticizer comprises water, ethanol, propylene carbonate, ethyl lactate, dimethyl acetamide or combinations thereof. These plasticizers are relatively cheap and provide a sufficient solvating environment.

It may further be particularly preferred that the plasticizer is suitable for spray-drying as this may allow for a scalable process. Typically, for a scalable process, the preferred plasticizer comprises one or more ICH class 3 solvents according (ICH) guideline Q3C (R6), established by the International Council for Harmonisation of Technical Requirements for Pharmaceuticals for Human Use. In particular the plasticizer preferably comprises one or more solvents within this class with a boiling point below 100.1° C., Accordingly, most preferably, the plasticizer comprises ethanol, isopropanol, ethyl acetate, tetrahydrofuran and/or methanol.

Providing a first DES of the API salt and the eutectic constituent according to the present invention is particularly suitable for API’s and salts thereof that are poorly soluble, especially in water and the aforementioned solvents for spray-drying. The poor solubility of such API’s and salts thereof does not only play a part in their solvation and bioavailability after administration to the patient, it also is relevant for the process of formulating these API’s and salts thereof into a suitable formulation. Conventionally, to liquify and render the API’s miscible, large amounts of solvents or unfavorable solvents must be used, if this is at all possible. By providing the first DES with the API salt, formulation of the API salt is much improved. Accordingly, the present invention is particularly suitable for API salts having a solubility of less than 20 mg/ml in the plastizer, preferably less than 20 mg/ml in water, ethanol, isopropanol, ethyl acetate, tetrahydrofuran, methanol and combinations thereof. At such low solubility, formulating such as spray-drying is practically and economically unfeasible without the present invention.

It is preferred that the first plasticizer is removed by evaporation using a suitable apparatus such as a rotary evaporator. The partial removal of the first plasticizer yields a solid DES that may contain a small amount of plasticizer. Preferably, the first plasticizer is removed up to 80% (m/m), in relation to the amount of plasticizer present in the first DES. Residual plasticizer may have a stabilizing effect on the solid DES in case the plasticizer is water, as water can be incorporated in the solid DES structure. Furthermore, if water is used as plasticizer, the ease of removal of the plastizicer typically i.a. depends on the hygroscopicity of the solid DES. For plasticizers that may not have a stabilizing effect on the solid DES, it may be preferred to remove the first plasticizer up to 90% (m/m), more preferably up to 95% (m/m), even more preferably up to 99% (m/m), most preferably essentially all or as much as possible. Removal of the plasticizer can lead to a chemically and/or physically stable state of the solid DES. More remaining plasticizer typically means more movement and thus a decrease in the chemical and physical stability of the solid DES. The amount of remaining plastizicer in the solid DES is than typically 23% (m/m) or less, preferably 18% (m/m), more preferably 14% (m/m) or less, even more preferably 10% (m/m) or less, most preferably 5% (m/m) or less, based on said solid DES.

Another constituent on which the solid DES according to the present invention is based is a pharmaceutically acceptable eutectic constituent (herein also referred to as the DES constituent). The DES constituent typically has the ability to create hydrogen bonds with, the API salt, as well as with the plasticizer to ensure a stable first DES is formed, e.g. typically hydroxyl groups are present. Moreover, the DES constituent preferably improves the bioavailablity of the API upon administration of the solid DES. Without wishing to be bound by theory, it is believed that the DES constituent may favorably increase the dissolution of the API in an aqueous environment. It is preferred that one single eutectic constituent is used to form a first DES. For example, the only eutectic constituent added may be urea or a cyclodextrin and not a combination of thereof. Having only one single eutectic constituent is advantageous as it allows for a higher drug loading and/or for enhanced physical stability.

The pharmaceutically acceptable eutectic constituent may comprise one or more carboxylic acids, phenolic compounds, terpenoids, organic bases, sugars, sweeteners, glycols, amino acids, quaternary ammonium compounds, derivatives of these classes and combinations thereof.

In a particular embodiment, the pharmaceutically acceptable eutectic constituent comprises one or more carboxylic acids. For instance the carboxylic acids may include, but are not limited to, malic acid, citric acid, lactic acid, fumaric acid, tartaric acid, ascorbic acid, pimelic acid, gluconic acid, acetic acid and/or derivatives thereof such as nicotinamide. However, carboxylic acids may influence the chemical stability of the constituents of the first DES and/or solid DES in particular the API salt. This is typically the case for API’s that are not acidic or organic acids by themselves. Accordingly, it is preferred that the eutectic constituent does not comprise a carboxylic acid and/or that the first DES and the solid DES are substantially free from an organic acid unless said API salt comprises an organic acid. Substantially free herein means that the first DES and the solid DES comprises less acid than would be detrimental to the allowable stability of the DES. Typcially, substantially free herein means that the first DES and the solid DES comprise less than 5%, preferably less than 1%, most preferably less than 0.1% of organic acid, based on the molar amount of API in the DES.

The pharmaceutically acceptable eutectic constituent may further or alternatively comprise one or more phenolic compounds that may include, but are not limited to, tyramine hydrochloride, tocopherol, butyl paraben and vanillin.

Additionally or alternatively, the pharmaceutically acceptable eutectic constituent may further comprise one or more terpenoids, which may be one of, but not limited to, terpineol, menthol and perillyl alcohol.

The organic bases that the pharmaceutically acceptable eutectic constituent may additionally or alternatively comprise may include, but are not limited to, urea and guanine.

In another embodiment the pharmaceutically acceptable eutectic constituent may further or alternatively comprise one or more sugars or sweeteners, that may include, but are not limited to, sucrose, glucose, fructose, lactose, maltose, xylose, sucrose, inositol, xylitol, saccharin, sucralose, aspartame, acesulfame potassium and ribotol, as well as their phosphates.

Additionally or alternatively, the pharmaceutically acceptable eutectic constituent may comprise one or more amino acids, these include, but are not limited to, cysteine, tyrosine, lysine, serine, glutamine, alanine and leucine. Preferably, the amino acid is a neutral amino acid.

In another embodiment, the pharmaceutically acceptable eutectic constituent may further or additionally comprise one or more quaternary ammonium compounds that include, but are not limited to, choline chloride, thiamine mononitrate and carnitine.

More generally, host-guest chemistry may be applicable, wherein the pharmaceutically acceptable eutectic constituent may be considered as host and the API as guest. Host-guest chemistry is a term typically used for supramolecular complexes in which two or more molecules or ions are held together by forces other than fully covalent bonds. These non-covalent interactions may include ionic bonding, hydrogen bonds, Van der Waals forces or hydrophobic interactions. The host usually comprises a void, core, cavity or a pocket, these terms are herein used interchangeably. The core relates to a suitable shape for a second molecule to position into. Molecules comprising such cores include, but are not limited to, cyclodextrins, cucurbiturils, calixarenes, catenanes, cryptands, crown ethers and pillararenes. Preferably, the exterior of the pharmaceutically acceptable eutectic constituent is hydrophilic and the core is hydrophobic. The hydrophobic core is generally capable of forming hydrophobic interactions with the API.

Preferably the pharmaceutically acceptable constituent comprises a sugar, more preferably a cyclodextrin.

Cyclodextrins usually comprise a hydrophilic exterior and a hydrophobic core, as described herein-above. They typically enable solvation of apolar compounds in polar solvents. The core may be tuned to be suitable for positioning and solvating the API. Tuning typically includes reducing or enlarging the size of the core, it moreover may include modification of the hydroxyl groups. Suitable cyclodextrins include, but are not limited to, alpha-, beta-, gamma-cyclodextrin, comprising respectively 6, 7, 8 glucose units.

Optionally the solid DES further comprises a polymeric precipitation inhibitor (PPI). PPIs may have high solubility (e.g. 200 mg/ml) in the plasticizer and therefore the plasticizer typically assists in solubilizing the PPI in the first DES. The mechanism of the PPI can vary from polymer to polymer but in general they create another phase for the API to be in other than a crystalline phase. Typically a supersaturated state is created after introducing the active ingredient into an aqueous environment (e.g. after administration of the solid DES) where its solubility is less. Generally, this supersaturated state gives rise to liquid-liquid phase separation (LLPS), thereby creating amorphous highly concentrated drug regions that, due to their metastable nature, crystallize over time. PPIs may create another phase for the API to be in other than the crystalline phase, thereby generally minimizing formation of large precipitant crystals. The maximum concentration in the aqueous environment of the active ingredient is typically several orders of magnitude lower than the maximum concentration in the solid DES. The PPIs used generally increase the solubility in ways through a metastable state, such that up to several orders of magnitude increase in solubility can be achieved, as well as smaller sizes of precipitant and lower rates of crystal growth. The addition of a PPI may further be beneficial for enhancing the bioavailability of the active ingredient.

Preferably, the PPI is a water-soluble polymer and may be selected from the group consisting of homopolymers and copolymers of N-vinyl lactams, especially homopolymers and copolymers of N-vinyl pyrrolidone, e.g. polyvinyl pyrrolidone (PVP), copolymers of N-vinyl pyrrolidone and vinyl acetate or vinyl propionate, polyvinyl caprolactam-polyvinyl acetate-polyethylene glycol graft copolymers, such as Soluplus®, block copolymers of ethylene oxide and propylene oxide, also known as polyoxyethylene / polyoxypropylene block copolymers or polyoxyethylene polypropyleneglycol, such as Poloxamer®, lauroyl polyoxyglycerides cellulose esters and cellulose ethers; in particular methylcellulose, hydroxyalkylcelluloses, in particular hydroxypropylcellulose, hydroxyalkylalkylcelluloses, in particular hydroxypropylmethylcellulose, high molecular polyalkylene oxides such as polyethylene oxide and polypropylene oxide and copolymers of ethylene oxide and propylene oxide, vinyl acetate polymers such as copolymers of vinyl acetate and crotonic acid, partially hydrolyzed polyvinyl acetate (also referred to as partially saponified “polyvinyl alcohol”), polyvinyl alcohol, oligo- and polysaccharides such as carrageenans, galactomannans and xanthan gum, and mixtures of one or more thereof.

Preferably the plasticizer, the pharmaceutically acceptable eutectic constituent and the optional PPI are broadly used in the pharmaceutical and/or food industry. Typically, this indicates that they are pharmaceutically acceptable at their relevant doses. More preferably the plasticizer, the pharmaceutically acceptable eutectic constituent and the optional PPI are generally recognized as safe and may be on the GRAS list for oral administration. A compound is typically considered GRAS after premarket review and approval by the FDA or if it has been considered generally safe amongst qualified experts and is has been adequately shown to be safe under the conditions for its intended use.

According to the present invention, addition of the first plasticizer to the solid DES results in the first DES. It is moreover possible to add a second plasticizer to the solid DES and yield a second DES. Herein, the second plasticizer and the second DES may be the same or different from the first plasticizer and the first DES. In other words, formation of a DES occurs after addition of a plasticizer to a solid DES regardless of what plasticizer was used to initially make the first DES. For example, a first DES with water as plasticizer is formed and after dehydration the solid DES is obtained. Hydration of the solid DES will reconstitute the second DES which is the same as the first DES (assuming the same amount of water is present). A second example is when a first DES with propylene carbonate is used to obtain the solid DES, hydration of the solid DES results in a second DES with water as main plasticizer, although residue propylene carbonate may be present. In the second example, the first and second DESs have a different composition.

In order to form a stable first and/or second DES the plasticizer is added typically up to 60% of the total mass of the first DES. Typically, the same amount of plasticizer that was removed from the first DES to form the solid DES may be added to reform a DES, i.e. the second DES.

The ability to reform a DES (i.e. the second DES) from the solid DES, is a property which distinghuises the solid DES from solid dispersions. The inventors have surprisingly found that even when the components of the solid DES and a solid dispersion may be the same, it may be that only the solid DES is capable of forming a DES. The solid dispersion may for instance merely disintegrate into a liquid dispersion. Without wishing to be bound by theory, the inventors believe that the formation of the first DES may form molecular interactions or arrangements that are beneficial for the reformation of the second DES after the first DES has been solidified by removing the first plasticizer.

Further, again without wishing to be bound by theory it is believed that in case the solid DES is orally administered the solid DES formulation does not immediately disintegrate in the aqueous environment but that instead after administration, the corresponding, second DES with the API salt as one of its constituents is formed. This corresponding DES is generally miscible with the aqueous environment, therefore gradually releasing the constituents of the mixture into the surrounding aqueous environment and preventing or limiting precipitation. Furthermore, an increased dissolution rate is typically observed due to the miscibility of the second DES with the environment. An increased dissolution rate may be beneficial for the bioavailability. The API may be absorbed by the patient following its release.

The present invention and its preferred embodiments thus differ from conventional solid dispersions as solid dispersions do not first create a DES or even a liquid before releasing the constituents in the surrounding aqueous environment after oral administration. Furthermore, as noted above, conventional solid dispersions do not create a DES after the addition of one or more plasticizers. If solid dispersions are in contact with plasticizers, dissolution and subsequent crystallization of the API typically occurs. Moreover, there is a general tendency of amorphous solid dispersions to go from amorphous to crystalline over time, even under storage conditions. Thus becoming physically unstable and thereby limiting its shelf life.

The solid DES according to the present invention has the benefits of a liquid formulation when administrated with the stability benefits of a solid formulation during storage. Crystallization of the API during storage of the solid DES is typically inhibited, even with significant amounts of plasticizer present. The physical and chemical stability of the solid DES are typically boosted as the molecular movement is reduced after removal of the plasticizer. In the present invention, the API salt itself is one of the DES constituents, in contrast to solid dispersions wherein the API is embedded in a polymer matrix. Moreover, the water compatibility of the solid DES mean that additions of small amount of water for example up to 10% do not negatively affect the physical stability of the solid DES thereby extending the shelf life.

The solid DES may be processed into a powder, granules, solid tablet or a combination thereof. Furthermore, the processed material may contain up to 18% (m/m) of the plasticizer, preferably up to 14% (m/m), more preferably up to 10% (m/m), most preferably up to 5% (m/m) in relation to the total mass of the solid DES. The processed material is potentially made into a suspension (e.g. for intravenous administration), encapsulated or pressed into tablets. The material may be used for medical treatment comprising enteral or intravenous administration, preferably enteral, more preferably oral administration. More preferably the solid DES is encapsulated and used for oral administration. After administration, the corresponding, second DES may be formed and release of the drug into the patient is typically realized. The bioavailability may be enhanced as the second DES is formed. The optional PPI generally prevents possible large crystals from being formed, therefore typically further increasing absorption of the API by the patient. The wide range of possibilities to tune the first DES and therefore the solid DES, may result in the possible incorporation of a wide variety of APIs.

For the purpose of clarity and a concise description features are described herein as part of the same or separate embodiments, however, it will be appreciated that the scope of the invention may include embodiments having combinations of all or some of the features described.

### Example 1

preferred example of a solid DES was formed consisting of an API X salt as API, hydroxylpropyl-B-cyclodextrin as pharmaceutically acceptable eutectic constituent and water as plasticizer. API X is a small molecule tyrosine kinase inhibitor and has a water solubility of less than 0.1 µg/ml in pH 7 water. The API X and hydroxylpropyl-B-cyclodextrin were mixed in a 1:1 molar ratio. To this mixture 50 wt% of water was added to form the first DES. Evaporation under vacuum at 40° C. overnight yielded the solid solid DES, leaving approximately 5% water. Herein, wt% relates to the total mass of added API plus added pharmaceutically acceptable eutectic constituent to create the first DES.

The black line with squares in FIG. 1 shows the resulting solid DES after in vitro dissolution. The in vitro dissolution occured in a simulated gastric medium of pH 1.6 for 30 minutes to simulate the stomach environment. The pH shift after 30 minutes occured by changing the medium to a simulated intestinal medium, with an pH of 6.5 to simulate the intestinal environment. It shows that the maximum concentration of API was approached before and after the pH-shift, thereby indicating proper solubility of API X in both the aqueous environments.

### Example 2

A preferred example of a solid DES was formed consisting of an API X salt as API, hydroxylpropyl-B-cyclodextrin as pharmaceutically acceptable eutectic constituent, Soluplus® (commercially available from BASF) as PPI and water as plasticizer. API X is a small molecule tyrosine kinase inhibitor and has a water solubility of less than 0.1 µg/ml in pH 7 water Soluplus® is a polyvinyl caprolactam-polyvinyl acetate-polyethylene glycol graft copolymer. The API X (232 mg) and hydroxylpropyl-B-cyclodextrin (568 mg) were mixed in a 1:1 molar ratio. To this mixture 500 µl (50% wt) of water was added to form the first DES. Subsequently, 200 mg (20 wt%) of Soluplus® was added. Evaporation under vacuum at 40° C. overnight yielded the solid solid DES with a total weight of approximately 1075 mg, leaving approximately 7.5 wt% water. Herein, wt% relates to the total mass of added API, added pharmaceutically acceptable eutectic constituent and added PPI to create the first DES.

The black line with triangles in FIG. 1 shows the resulting solid DES after in vitro dissolution. The in vitro dissolution occured in a simulated gastric medium of pH 1.6 for 30 minutes to simulate the stomach environment. The pH shift after 30 minutes occured by changing the medium to a simulated intestinal medium, with an pH of 6.5 to simulate the intestinal environment. It shows that the maximum concentration of API was approached before and after the pH-shift, thereby indicating proper solubility of API X in both the aqueous environments.

Further, FIG. 2 shows a comparative in vitro dissolution of this solid DES formulation (X- Formulation) compared to the crystalline free base form of the API X and the crystalline mesylate salt form of the API. The black bars indicate the API concentration in pH 1.6 fasted state biorelevant gastric media after 30 minutes. The grey bars indicate the API concentration in pH 6.5 fasted state biorelevant intestinal media after 60 minutes. The formulation was added to the gastric media and after 30 minutes intestinal media was added to the sample. The lines on the graph show the theoretical maximum concentration the formulation could achieve in the experiment. This data shows a 12.5x increase in solubility of the API in intestinal media after 60 minutes of dissolution compared to the crystalline mesylate salt and a 137.4x increase compared to the free base.

Further still, FIGS. 5 and 6 are polarized light microscopy images of the formed solid DES that illustrate that no crystals are present and thus that the solid DES is a glassy solid and not a crystalline material.

### Example 3

A preferred example of a solid DES was formed consisting of an API Y mesylate salt as API, hydroxylpropyl-B-cyclodextrin as pharmaceutically acceptable eutectic constituent, Soluplus® (commercially available from BASF) as PPI and water as plasticizer. Soluplus® is a polyvinyl caprolactam-polyvinyl acetate-polyethylene glycol graft copolymer. The API Y mesylate salt (192.4 mg) and hydroxylpropyl-B-cyclodextrin (607.6 mg) were mixed in a 1:1.5 molar ratio. To this mixture 240 µl (30% wt) of water was added to form the first DES. Subsequently, 200 mg (20 wt%) of Soluplus® was added. Evaporation under vacuum at 40° C. overnight yielded the solid DES with a total weight of approximately 1030 mg, leaving approximately 3 wt% water. Herein, wt% relates to the total mass of added API, added pharmaceutically acceptable eutectic constituent and added PPI to create the first DES.

The composition prepared showed a purity of 99.32% when measured on UPLC, showing no measurable decrease in chemical stability when stored at 40° C. for 14 days.

FIG. 3 shows a comparative in vitro dissolution of this solid DES formulation (Y-Formulation) compared to the crystalline free base form of the API Y and the crystalline mesylate salt form of the API. The black bars indicate the API concentration in pH 1.6 fasted state biorelevant gastric media after 30 minutes. The grey bars indicate the API concentration in pH 6.5 fasted state biorelevant intestinal media after 60 minutes. The formulation was added to the gastric media and after 30 minutes intestinal media was added to the sample. The lines on the graph show the theoretical maximum concentration the formulation could achieve in the experiment. This data shows a 12.5x increase in solubility of the API in intestinal media after 60 minutes of dissolution compared to the crystalline mesylate salt and a 25x increase compared to the free base.

### Comparative Example 1

The same procedure as described above for Example 3 was followed, except API Y and methanesulfonic acid were mixed in situ_in the following procedure.

The API Y (144.4 mg), methanesulfonic acid (48.0 mg) and hydroxylpropyl-B-cyclodextrin (607.6 mg) were mixed in a 1:1:1.5 molar ratio. To this mixture 240 µl (30% wt) of water was added to form the first DES. Subsequently, 200 mg (20 wt%) of Soluplus® was added. Evaporation under vacuum at 40° C. overnight yielded the solid DES with a total weight of approximately 1030 mg, leaving approximately 3 wt% water. Herein, wt% relates to the total mass of added API, added pharmaceutically acceptable eutectic constituent and added PPI to create the first DES.

The composition prepared with the mesylate salt showed a purity of 99.32% when measured on UPLC, and a purify of 95.25% after storage at 40° C. for 14 days. This shows a decrease in chemical stability of 4.07%.

### Example 4

A preferred example of a solid DES was formed consisting of an itraconazole HCl salt as API, hydroxylpropyl-B-cyclodextrin as pharmaceutically acceptable eutectic constituent, Soluplus® (commercially available from BASF) as PPI and water as plasticizer. Soluplus® is a polyvinyl caprolactam-polyvinyl acetate-polyethylene glycol graft copolymer. The API (247.3 mg) and hydroxylpropyl-B-cyclodextrin (452.7 mg) were mixed in a 1:1 molar ratio. To this mixture 280 µl (40% wt) of water was added to form the first DES. Subsequently, 300 mg (30 wt%) of Soluplus® was added and solubilized until a transparent liquid was formed. Evaporation under vacuum at 40° C. overnight yielded the solid DES with a total weight of approximately 1050 mg, leaving approximately 5 wt% water. Herein, wt% relates to the total mass of added API, added pharmaceutically acceptable eutectic constituent and added PPI to create the first DES.

FIG. 4 shows a comparative in vitro dissolution of this solid DES formulation (P38) compared to the crystalline free base form of the API (ITZ) and the crystalline HCl salt form of the API. The black bars indicate the API concentration in pH 1.6 fasted state biorelevant gastric media after 30 minutes. The grey bars indicate the API concentration in pH 6.5 fasted state biorelevant intestinal media after 60 minutes. The formulation was added to the gastric media and after 30 minutes intestinal media was added to the sample. This data shows a 16.2x increase in solubility of the API in intestinal media after 60 minutes of dissolution compared to the crystalline HCl salt and a 86.5x increase compared to the free base.

### Example 5

A preferred example of a solid DES was formed consisting of an itraconazole HCl salt as API, citric acid as pharmaceutically acceptable eutectic constituent. Soluplus® (commercially available from BASF) as PPI and water as plasticizer. Soluplus® is a polyvinyl caprolactam-polyvinyl acetate-polyethylene glycol graft copolymer. The API (401.1 mg) and citric acid (198.9 mg) were mixed in a 1:2 molar ratio. To this mixture 240 µl (40% wt) of water was added to form the first DES. Subsequently, 400 mg (40 wt%) of Soluplus® was added and solubilized until a transparent liquid was formed. Evaporation under vacuum at 40° C. overnight yielded the solid DES with a total weight of approximately 1060 mg, leaving approximately 6 wt% water. Herein, wt% relates to the total mass of added API, added pharmaceutically acceptable eutectic constituent and added PPI to create the first DES.

FIG. 4 shows a comparative in vitro dissolution of this solid DES formulation (P24) compared to the crystalline free base form of the API ITZ) and the crystalline HCl salt form of the API. The black bars indicate the API concentration in pH 1.6 fasted state biorelevant gastric media after 30 minutes. The grey bars indicate the API concentration in pH 6.5 fasted state biorelevant intestinal media after 60 minutes. The formulation was added to the gastric media and after 30 minutes intestinal media was added to the sample.. This data shows a 6.07x increase in solubility of the API in intestinal media after 60 minutes of dissolution compared to the crystalline HCl salt and a 32.4x increase compared to the free base.

Further, FIG. 7 shows four differential scanning calorimetery thermograms that indicate that the solid DES formulation (P24) lacks crystalline material. The citric acid and itraconazole thermal events indicate that melting is occurring in the individual consitituents but when they are formulated into a solid DES no thermal events occur. This indicates the solid DES is a glassy solid eutectic mixture.

### Example 6

Sildenafil citrate (100 mg) was put in an polypropylene container. Urea (150 mg) was added and the two solids were mixed.

Ethanol (100 uL) and water (100 uL) were added and the mixture was agitated for 10 minutes at 60° C. until a clear liquid was formed.

500 uL of a liquid of 143 mg/g polyvinylpyrrolidone K-90 in a mixture of 1:1 water:ethanol (v/v) was added and the mixture was agitated until homogenized, as illustrated in FIG. 8.

The mixture was concentrated in vacuo to yield a clear solid. Under polarized light no crystalized material was visible (see FIG. 9).

### Comparative Example 2

The same procedure as described above for Example 6 was followed, except the urea eutectic constituent being omitted. This control sample did not yield a clear liquid as can be seen in FIG. 8 and evaporation of ethanol and water yielded a turbid solid. Under polarized light a significant amount of crystalized material was visible (see FIG. 10).

### Example 7

Sildenafil citrate (100 mg) was put in an polypropylene container. Ascorbic acid (150 mg) was added and the two solids were mixed.

Ethanol (100 uL) and water (200 uL) were added and the mixture was agitated for 10 minutes at 60° C. until a clear solution was formed.

500 uL of a solution of 143 mg/g polyvinylpyrrolidone K-90 in a mixture of 1:1 water:ethanol (v/v) was added and the mixture was agitated until homogenized, as illustrated in FIG. 11.

The mixture was concentrated in vacuo to yield a clear solid. Under polarized light no crystalized material was visible (see FIG. 12).

### Comparative Example 3

The same procedure as described above for Example 7 was followed, except the ascorbic acid eutectic constituent being omitted. This control sample did not yield a clear solution as can be seen in FIG. 11 and evaporation of ethanol and water yielded a turbid solid. Under polarized light a significant amount of crystalized material was visible (see FIG. 13).

### Example 8

Fexofenadine (50 mg) was put in an polypropylene container. Meglumine (100 mg) was added and the two solids were mixed.

Ethanol (200 uL) and water (500 uL) were added and the mixture was agitated for 10 minutes at 60° C. until a clear solution was formed.

500 uL of a solution of 143 mg/g polyvinylpyrrolidone K-90 in a mixture of 1:1 water:ethanol (v/v) was added and the mixture was agitated until homogenized (see FIG. 14).

The mixture was concentrated in vacuo to yield a clear solid. Under polarized light no crystalized material was visible (see FIG. 15).

### Comparative Example 4

The same procedure as described above for Example 8 was followed, except the meglumine eutectic constituent being omitted. This control sample did not yield a clear solution as illustrated in FIG. 14 and evaporation of solvents yielded a turbid solid. Under polarized light a significant amount of crystalized material was visible (see FIG. 16).

1. A method to prepare a solid deep eutectic solvent (solid DES) that is solid at 20° C., said method comprising providing a first DES and at least partially removing a first plasticizer from said first DES, wherein said first DES is at least based on said first plasticizer, an active pharmaceutical ingredient (API) salt, a pharmaceutically acceptable eutectic constituent and optionally a polymeric precipitation inhibitor (PPI).

2. The method according to claim 1, wherein the pharmaceutically acceptable eutectic constituent is selected from the group consisting of phenolic compounds, terpenoids, organic bases, sugars, sweeteners, glycols, amino acids, quaternary ammonium compounds, and combinations thereof and wherein the first DES and the solid DES are substantially free from an organic acid unless said API salt comprises an organic acid.

3. The method according to claim 1, wherein providing said first DES comprises providing the API salt and mixing said API salt with the pharmaceutically acceptable eutectic constituent and the plasticizer and optionally the PPI.

4. The method according to claim 1, wherein the the plasticizer is selected from the group consisting of water, ethanol, isopropanol, ethyl acetate, tetrahydrofuran, methanol and combinations thereof, and wherein the saturated loading of the API salt in the DES is greater than the saturated loading of the API salt in the same volume of the used plasticizer.

5. The method according to claim 1, wherein the solid DES is solid at 30° C.

6. The method according to claim 1, wherein the solid DES comprises the first plasticizer in an amount of less than 23% (m/m), based on the mass percentage of the plasticizer in said solid DES.

7. The method according to claim 1, wherein said first and/or second plasticizer functions as hydrogen bond donor and/or acceptor.

8. The method according to claim 1, wherein said first and/or second plasticizer comprises water, propylene carbonate, ethyl lactate, dimethyl acetamide, ethanol or combinations thereof.

9. The method according to claim 1, wherein the amount of the API salt in the first DES is more than would be soluble in its isolated form in the amount of the first plasticizer that is used in the first DES.

10. The method according to claim 1, wherein the weight ratio of the API salt to the pharmaceutically acceptable eutectic constituent is such that the API salt and the pharmaceutically acceptable eutectic constituent form a eutectic mixture.

11. The method according to claim 1, wherein the solid DES comprises the API salt in an amount of more than 5 wt% based on the weight of the solid DES.

12. The method according to claim 1, wherein the pharmaceutically acceptable eutectic constituent comprises one or more carboxylic acids, phenolic compounds, terpenoids, organic bases, sugars, sweeteners, glycols, amino acids, quaternary ammonium compounds, derivative of these classes or combinations thereof.

13. The method according to claim 1, wherein the first DES comprises one single pharmaceutically acceptable eutectic constituent.

14. The method according to claim 1, wherein said PPI is selected from the group consisting of homopolymers and copolymers of N-vinyl lactams, especially homopolymers and copolymers of N-vinyl pyrrolidone, polyvinyl caprolactam-polyvinyl acetate-polyethylene glycol graft copolymers, block copolymers of ethylene oxide and propylene oxide, also known as polyoxyethylene / polyoxypropylene block copolymers or polyoxyethylene polypropyleneglycol, lauroyl polyoxyglycerides cellulose esters and cellulose ethers.

15. The method according to claim 1 wherein the at least partially removing of said first plasticizer comprises evaporation.

16. The method according to claim 1, wherein said first plasticizer is for at least 80% (m/m) removed from said first DES, based on the amount of plasticizer in the first DES.

17. A solid DES that is solid at 20° C., comprising an API, a pharmaceutically acceptable eutectic constituent and optionally a first plasticizer and/or polymeric precipitation inhibitor (PPI), wherein said solid DES is obtainable by a process according to claim 1.

18. The solid DES according to claim 17, wherein said solid DES is processed into a powder, granulates, tablet or a combination thereof.

19. The solid DES according to claim 17, comprising the first plasticizer in an amount of up to 23% (m/m) based on said solid DES.

20. The solid DES according to claim 17, wherein the solid DES is solid at 30° C.

21. The solid DES according to claim 17 for use in a medical treatment comprising enteral or intravenous administration.
